(19) United States
(12) Patent Application Publication
(10) Pub. No.: US 2012/0077258 A1
(43) Pub. Date: Mar. 29, 2012

(54) DIAGNOSTIC DEVICE

(76) Inventors: Stefan Förtsch, Kunreuth (DE); Rainer Kuth, Hochstadt (DE); Karl-Heinz Maier, Nurnberg (DE)

(21) Appl. No.: 13/146,701

(22) PCT Filed: Feb. 15, 2010
(86) PCT No.: PCT/EP2010/051851

§ 371 Date: Jul. 28, 2011

(30) Foreign Application Priority Data
Feb. 17, 2009 (DE) 10 2009 009 292.7
May 28, 2009 (DE) 10 2009 023 035.1
Feb. 5, 2010 (DE) 10 2010 006 972.8

(51) Int. Cl. C12M 1/42

(52) U.S. Cl. 435/287.1

(57) ABSTRACT

A diagnostic device as a first electrode formed by a noble metal that is not attackable by acid, and a second electrode that is formed of silver. The first and second electrodes are at least partially immersed in a nutrient solution contained in a container, into which a tissue sample can be introduced. An electrical voltage is applied between the first and second electrodes, and a change in an electrical variable between the electrodes is measured when ammonia is present. The diagnostic device allows fast screening of tissue samples for Helicobacter pylori.

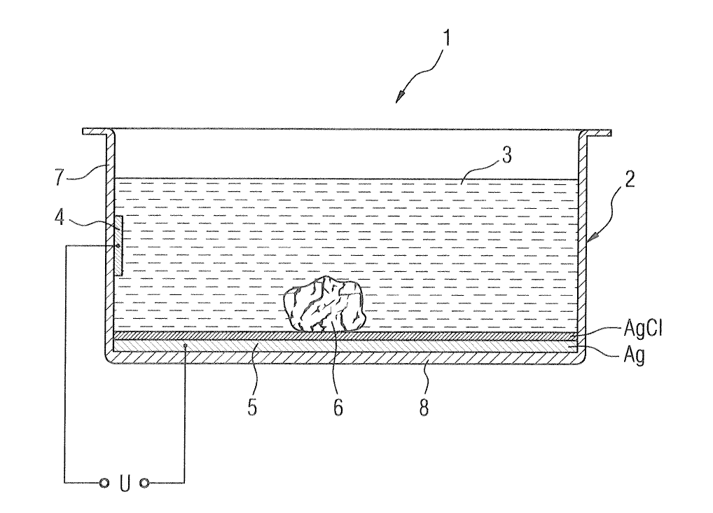

### BACKGROUND OF THE INVENTION

1. Field of the Invention

The invention relates to a diagnostic device.

Such a diagnostic device serves, for example, for detecting Helicobacter pylori.

2. Description of the Prior Art

A common cause for discomforts in the upper gastrointestinal tract is a bacterial affliction of the organs therein. By way of example, an affliction with Helicobacter pylori is held responsible for a vast range of gastric disorders that go hand-in-hand with an increased secretion of gastric acid. By way of example, these include type B gastritis, approximately 75% of gastric ulcers and almost all duodenal ulcers. Hence, examining the hollow organs of the gastrointestinal tract for bacteria populations, more particularly Helicobacter pylori populations, is an important component for diagnosing gastric disorders.

For example, Helicobacter pylori is detected using a breath test, in which a patient is administered C-13 masked urea. The C-13 masked CO₂, which is created when urea (CO(NH₂)₂) is split into ammonia (NH₃) and carbon dioxide (CO₂), is detected in the exhaled air. Other methods for detecting Helicobacter pylori are directed at typical blood values such as pepsinogen or gastrin. However, such methods are complex and only have limited reliability. A further test for Helicobacter pylori is the detection of the Helicobacter pylori antigen in fecal matter.

A further option for examining the stomach for a Helicobacter pylori population is provided by so-called gastroscopy. During such an examination, a gastroenterologist takes a tissue sample (biopsy specimen) from the mucosa of the stomach by means of a biopsy in order to examine, either immediately or at a later stage, whether there is an infection with Helicobacter pylori. A known examination method for the tissue sample is, for example, the Helicobacter urease test (HU test, abbreviated HUT). The biopsy specimen is placed into a test medium (measurement solution), which consists of a nutrient solution for this bacteria, urea, and an indicator (litmus). If Helicobacter pylori bacteria is contained in the sample, the bacteria splits the urea (CO(NH₂)₂) using urease into ammonia (NH₃) and carbon dioxide (CO₂). The ammonia then colors the indicator red. The test result is ready after a few minutes. The onset of color change from yellow to red cannot unambiguously be identified in inexpedient conditions.

An alternative to gastroscopy carried out using a flexible endoscope is to use a so-called endoscopic capsule. Such an endoscopic capsule, which is also referred to as a capsule endoscope or endocapsule, is embodied as a passive endocapsule or a navigable endocapsule. A passive endoscopic capsule moves through the intestines of the patient as a result of peristalsis.

For example, a navigable endocapsule is known from DE 101 42 253 C1 and the corresponding patent application with the publication number US 2003/0060702 A1, and therein it is referred to as an “Endoroboter” or “endo-robot”. The endo-robot known from these publications can be navigated in a hollow organ (e.g. gastrointestinal tract) of a patient by means of a magnetic field, which is generated by an external (i.e. arranged outside of the patient) magnetic system (coil system). An integrated system for controlling the position, which comprises a positional measurement of the endo-robot and automatic regulation of the magnetic field or the coil currents, can be used to detect changes automatically in the position of the endo-robot in the hollow organ of the patient and to compensate for these. Furthermore, the endo-robot can be navigated to desired regions of the hollow organ in a targeted fashion. It is for this reason that this type of capsule endoscopy is also referred to as magnetically guided capsule endoscopy (MGCE).

### SUMMARY OF THE INVENTION

An object of the present invention is to provide a diagnostic device that can be used to test a fresh tissue sample for Helicobacter pylori within a very short period of time.

The diagnostic device according to the invention has a first electrode (reference electrode) made of a noble metal, which cannot be attacked by acid (e.g. hydrochloric acid, phosphoric acid, sulfuric acid, gastric acid), and a second electrode (measurement electrode) made of silver. The first electrode and the second electrode are at least partially immersed in a container that is filled with a nutrient solution (measurement solution) and into which a tissue sample can be introduced. An electric voltage can be applied between the first electrode and the second electrode, and a change in an electric variable can be measured if ammonia is present between the first electrode and the second electrode.

In a preferred version of the diagnostic device according to the invention, the electric voltage between the first electrode and the second electrode equals zero. Thus no current flows between the first electrode and the second electrode. The potential thus is measured (i.e. without a current) between the first electrode and the second electrode. Thus there is barely any ionic migration in the acidic nutrient solution. In a further advantageous embodiment, the electric voltage between the first electrode and the second electrode is an AC voltage with a variably predeterminable frequency spectrum. If nutrient solution is exposed to direct current or a directed potential the ions migrate to the associated electrodes, i.e. the cations (e.g. ammonium NH₄⁺) migrate to the cathode and the anions (e.g. chloride Cl⁻) migrate to the anode. By applying a suitable AC voltage, the diagnostic device according to claim 3 reliably prevents complete charging of the first electrode (reference electrode) and complete charging of the second electrode (measurement electrode) because the migration speed of the ions in the acidic nutrient solution (measurement solution) is almost zero if the frequency is sufficiently high.

When an AC voltage is applied, there is a cyclical change at the second electrode (measurement electrode), which, according to the invention, consists of silver (Ag), between destruction and buildup of the silver chloride (AgCl) layer. Both the destruction of the silver chloride layer and the buildup thereof can be measured by e.g. an impedance measurement and can be compared cyclically. The potential differences and phase differences that can be measured in the process are characteristic for the presence of urease activity, as a result of which presence of Helicobacter pylori can be deduced with a very high certainty.

In a further embodiment, the frequency spectrum of the AC voltage is modulated. As a result, a higher AC voltage stability is obtained, which increases the measurement accuracy and reduces the measurement duration.

Alternatively, the electric voltage between the first electrode and the second electrode is a DC voltage, which can be applied for a predeterminable period of time. The predeterminable period of time during which an electric voltage can be applied by the user between the first electrode and the second electrode may lie between zero seconds and continuously, wherein the electric voltage selected by the user may be zero volts or higher. In the case of a period of time of zero seconds or a voltage of zero volts, this is a passive measurement. In the case of values deviating from these, this is an active measurement.

In further embodiments of the diagnostic device according to the invention, e.g. potentials, electric currents or electric resistances or the changes therein or variables (e.g. electric conductivity) derived from the electric variables or changes therein can be measured as electric variables.

The second electrode (measurement electrode), which consists of silver (Ag) in diagnostic device according to the invention, must be etched (roughened) by hydrochloric acid (HCl). This may (but this is not necessary) already occur for the first time before the diagnostic device or the second electrode is supplied. However, it is also possible for the users themselves to undertake the initial HCl etching or apply an appropriate silver chloride layer by means of a suitable electrolytic method. After HCl etching or after electrolytic deposition, the second electrode has a silver chloride (AgCl) coating on its surface and is therefore activated for the measurement to detect Helicobacter pylori.

The diagnostic device according to the invention allows simple open or closed loop control of the sensor or its first electrode (reference electrode) and/or its second electrode (measurement electrode) e.g. by means of a baseline correction. Furthermore, a reproducible regeneration of the second electrode, i.e. removal of the damage caused by ammonia in the silver chloride layer, is possible after each examination.

If the measures outlined above are taken, the second electrode is not completely charged, and so a regeneration of the second electrode only becomes necessary after a multiplicity of examinations.

Moreover, the sensitivity of the sensor and/or its first and/or second electrode can be set in a simple fashion in the diagnostic device according to the invention. The sensitivity can be set before and during the analysis in respect of Helicobacter pylori.

Platinum (Pt) and gold (Au) can be used as noble metals that are not attacked by acid and therefore are suitable for the first electrode (reference electrode).

Preferably, the nutrient solution (measurement solution) provided is an acidic nutrient solution, more particularly a hydrochloric nutrient solution. A buffered nutrient solution is particularly preferred. According to a further preferred embodiment, the urea is added to acidic nutrient solution.

If, then, a tissue sample taken from the gastro-intestinal tract is introduced into the hydrochloric nutrient solution (pH similar to that of the stomach), then affliction of the tissue sample with Helicobacter pylori can be detected by detecting ammonia (NH₃). Ammonia is generated by the Helicobacter pylori bacteria by splitting urea using urease in order to protect itself from the acidic environment of the gastrointestinal tract, more particularly the high acid concentration in the stomach.

As noted above, second electrode (measurement electrode), which consists of silver (Ag) in the diagnostic device according to the invention, must be etched by hydrochloric acid (HCl). After the HCl etching, the second electrode has a silver chloride (AgCl) coating on its surface and is therefore activated for the measurement to detect Helicobacter pylori. The activation of the second electrode is based on the following chemical reaction:

Ag+HCl→AgCl+H⁺+e⁻

Since ammonia (NH₃) under normal circumstances does not occur, or only occurs in very low concentrations in a hollow organ of the gastrointestinal tract, such as the stomach, as a result of the following neutralization reaction (forming an ammonium cation by protonation of ammonia)

NH₃+H⁺ [symbol] NH₄⁺

the detection thereof is a very strong indication for the presence of Helicobacter pylori. The proton (H⁺, hydrogen nucleus) is a component of the gastric acid.

The corresponding chemical reaction for detecting Helicobacter pylori is:

AgCl+2NH₃→[Ag(NH₃)₂]⁺+Cl⁻

The AgCl salt (silver chloride) is split into the silver-diammine complex [Ag(NH₃)₂]⁺ and chloride Cl⁻ by ammonia. [Ag(NH₃)₂]⁺ as a cation is very soluble in water and absorbed by the nutrient solution (measurement solution). In advantageous embodiments of the diagnostic device according to the invention, there is, between the first electrode (reference electrode) and second electrode (measurement electrode), either an electric voltage of zero or an electric AC voltage with a variably predeterminable frequency spectrum. Alternatively, a DC voltage can be applied between the first electrode and the second electrode for a predeterminable period of time. In all cases, there is barely any ion migration in the nutrient solution (migration speed of the cations and anions is approximately zero).

The electric variable (potential, electric current, electric resistance) measured between the first electrode (reference electrode) and second electrode (measurement electrode) is recorded, displayed, and—if desired—transmitted to evaluation electronics. As a result of an (automated) comparison between the measured value and predetermined values, a possible affliction of the mucosa of the stomach with Helicobacter pylori can be reliably indicated.

After the analysis of the tissue sample taken is completed, the container and the electrodes are first disinfected with a cleaning solution (e.g. ethanol or isopropanol) and subsequently rinsed with rinsing solution (hydrochloric acid or a mixture of hydrochloric acid and urea). By rinsing the second electrode (measurement electrode) with hydrochloric acid, the AgCl surface on the second electrode is regenerated. The damage to the AgCl layer of the second electrode caused by ammonia is thereby removed again. The diagnostic device according to the invention can thus once again be used for detecting Helicobacter pylori after refilling the container with a nutrient solution, preferably with an acidic nutrient solution, more particularly with a buffered nutrient solution, and after a possible necessary recalibration. By way of example, the diagnostic device can be calibrated by a dose of synthetic ammonia.

The diagnostic device according to the invention thus allows a very quick examination of tissue samples taken in respect of Helicobacter pylori.

Both the first electrode (reference electrode) and the second electrode (measurement electrode) may be embodied as separate rod-shaped or planar electrodes, which are at least in part immersed into the nutrient solution.

When the diagnostic device is used, care has to be taken that the nutrient solution at all times only wets the silver chloride layer, particularly even once the tissue sample has been introduced. However, as per an embodiment with a simple design of the diagnostic device according to the invention, the first electrode is integrated into a wall of the container or formed by a wall of the container. Additionally, or as an alternative thereto, the second electrode is formed by the base of the container as a further design simplification. A tissue sample taken from the patient can then be introduced into the nutrient solution anywhere within the container. As a result, direct positioning on the second electrode is not required.

### BRIEF DESCRIPTION OF THE DRAWINGS

The single FIGURE schematically illustrates a diagnostic device constructed and operating in accordance with the present invention.

### DESCRIPTION OF THE PREFERRED EMBODIMENTS

The invention and further advantageous embodiments are explained in more detail below in the drawing on the basis of a schematically illustrated exemplary embodiment; however, the invention is not restricted to the explained exemplary embodiment.

The only FIGURE shows a diagnostic device 1, which includes a container 2 which is filled with an acidic, preferably buffered nutrient solution 3 (measurement solution). In the illustrated exemplary embodiment, urea is added to the nutrient solution 3.

In the illustrated exemplary embodiment, the diagnostic device 1 has a first electrode 4 (reference electrode) made of a noble metal, which cannot be attacked by hydrochloric acid, and a second electrode 5 (measurement electrode) made of silver (Ag). The second electrode 5 has a silver chloride layer (AgCl layer) on its surface and is therefore activated for the measurement to detect Helicobacter pylori.

Platinum (Pt) and gold (Au) can be used as noble metals that are not attacked by hydrochloric acid and therefore are suitable for the first electrode 4.

The first electrode 4 and the second electrode 5 are at least partly immersed into the container 2.

A tissue sample 6 (biopsy specimen), which was taken from the mucosa of the stomach by means of a biopsy, has been introduced into the container 2 filled with the nutrient solution 3.

An electric voltage U can be applied between the first electrode 4 and the second electrode 5 for a predeterminable time, as a result of which a change in an electric variable, e.g. potential, electric current, or electric resistance, can be measured if ammonia is present between the first electrode 4 and the second electrode 5.

In the illustrated exemplary embodiment of the diagnostic device according to the invention, the first electrode 4 (reference electrode) is integrated into a wall 7 of the container 2 and the second electrode 5 (measurement electrode) is formed by the base 8 of the container 2. Hence, the illustrated embodiment of the diagnostic device according to the invention has a particularly simple design. The tissue sample 6 taken from the patient can then advantageously be introduced into the acidic nutrient solution 3 anywhere within the container 2. As a result, direct positioning of the tissue sample 6 on the second electrode 5 is not required.

Although modifications and changes may be suggested by those skilled in the art, it is the intention of the inventors to embody within the patent warranted heron all changes and modifications as reasonably and properly come within the scope of their contribution to the art.

1. A diagnostic device, comprising a first electrode (4) made of a noble metal, which cannot be attacked by acid, and a second electrode (5) made of silver, wherein the first electrode (4) and the second electrode (5) are at least partially immersed in a container (2) which is filled with a nutrient solution (3) and into which a tissue sample (6) can be introduced, and wherein an electric voltage (U) can be applied between the first electrode (4) and the second electrode (5), and a change in an electric variable can be measured if ammonia is present between the first electrode (4) and the second electrode (5).

2. The diagnostic device as claimed in claim 1, wherein the electric voltage (U) between the first electrode (4) and the second electrode (5) equals zero.

3. The diagnostic device as claimed in claim 1, wherein the electric voltage (U) between the first electrode (4) and the second electrode (5) is an AC voltage with a variably predeterminable frequency spectrum.

4. The diagnostic device as claimed in claim 1, wherein the electric voltage (U) between the first electrode (4) and the second electrode (5) is a DC voltage, which can be applied for a predeterminable period of time.

5. The diagnostic device as claimed in claim 1, wherein a potential can be measured as electric variable.

6. The diagnostic device as claimed in claim 1, wherein an electric current can be measured as electric variable.

7. The diagnostic device as claimed in claim 1, wherein an electric resistance can be measured as electric variable.

8. The diagnostic device as claimed in claim 1, wherein an acidic nutrient solution is provided as nutrient solution (3).

9. The diagnostic device as claimed in claim 8, wherein a hydrochloric nutrient solution is provided as acidic nutrient solution (3).

10. The diagnostic device as claimed in claim 1, wherein a buffered nutrient solution is provided as acidic nutrient solution (3).

11. The diagnostic device as claimed in claim 1, wherein urea is added to the acidic nutrient solution (3).

12. The diagnostic device as claimed in claim 1, wherein the first electrode (4) consists of platinum or of gold.

13. The diagnostic device as claimed in claim 1, wherein the first electrode (4) is integrated into a wall (7) of the container (2) or formed by a wall (7) of the container (2).

14. The diagnostic device as claimed in claim 1 or 2, wherein the second electrode (5) is formed by the base (8) of the container (2).

15. The diagnostic device as claimed in claim 1, wherein the second electrode (5) has a silver chloride layer.

16. The diagnostic device as claimed in claim 1, wherein the first electrode (4) and/or the second electrode (5) can be replaced.

17. The diagnostic device as claimed in claim 1, wherein the second electrode (5) can be regenerated.

18. The diagnostic device as claimed in claim 3, wherein the frequency spectrum of the AC voltage comprises pulses in the form of a sinusoidal voltage.

19. The diagnostic device as claimed in claim 3, wherein the frequency spectrum of the AC voltage comprises pulses in the form of a triangular voltage.

20. The diagnostic device as claimed in claim 3, wherein the frequency spectrum of the AC voltage comprises pulses in the form of a saw-tooth-shaped voltage.

21. The diagnostic device as claimed in claim 3, wherein the frequency spectrum of the AC voltage comprises a noise spectrum.

22. The diagnostic device as claimed in claim 3 or one of claims 18 to 21, wherein the frequency spectrum of the AC voltage comprises at least two pulses with different shapes.

23. The diagnostic device as claimed in claim 3 or one of claims 18 to 22, wherein the frequency spectrum of the AC voltage has components with different bandwidths.

24. The diagnostic device as claimed in claim 3 or one of claims 18 to 23, wherein the frequency spectrum of the AC voltage is modulated.
